(19) United States
(12) Patent Application Publication
(10) Pub. No.: US 2009/0084377 A1
(43) Pub. Date: Apr. 2, 2009

(54) ENDOTRACHEAL TUBE PROTECTOR

(76) Inventor: Julius Hajgato, Shanty Bay (CA)

Correspondence Address: NIKOLAI & MERSEREAU, P.A., 900 SECOND AVENUE SOUTH, SUITE 820, MINNEAPOLIS, MN, 55402, US

(21) Appl. No.: 11/865,411

(22) Filed: Oct. 1, 2007

(51) Int. Cl. A61M 16/04

(52) U.S. Cl. 128/200.26; 128/207.14

(57) ABSTRACT

A endotracheal tube protector for use with an endotracheal tube comprises an elongated tubular member having a first end configured to extend out of a patient's mouth when in use and a second end configured to enter the throat of the patient. The tubular member has openings in the first and second ends and defines a substantially cylindrical cavity with an interior wall configured to hold an endotracheal tube therewithin. The tubular member has a longitudinal slit for inducing radial expansion of the cavity and a pair of opposed bosses formed longitudinally in the tubular member at positions adjacent to the slit. The opposed bosses are configured to project into the cavity and to transmit a clamping force onto the endotracheal tube when contacted by the upper teeth of the patient when the endotracheal tube protector is in use and oriented such that the slit faces toward the roof of the patient's mouth.

### FIELD OF THE INVENTION

The present invention relates to medical equipment, specifically to endotracheal tube protectors, and more particularly to an endotracheal tube protector configured to secure endotracheal tubes of different diameters through clamp forces provided by the jaw of a patient when the endotracheal tube protector is in use.

### BACKGROUND OF THE INVENTION

Endotracheal tubes are used to provide a clear passage for air to the lungs in cases of injury or airway blockage or to provide a passage through which medical probes or instruments can be inserted such that damage of the tissues of the patient is minimized. Such endotracheal tubes often are used in conjunction with an air tube that leads to an inflatable bulb which functions to seal the space between the tracheal wall and the endotracheal tube so that saliva and/or other bodily fluids are prevented from entering the lungs.

Endotracheal tube protectors are used to prevent the patient from biting and damaging endotracheal tubes when in use, and for protecting the delicate tissues of the patient from abrasions caused by entry of the endotracheal tube into the oral cavity. Improvements in the design and function of endotracheal tube protectors for endotracheal tubes are needed. The present invention satisfies this need.

### SUMMARY OF THE INVENTION

It is an object of the present invention to provide an endotracheal tube protector for an endotracheal tube capable of accommodating endotracheal tubes of different sizes, while lessening the discomfort experienced by the intubated patient. This object may be realized by providing an endotracheal tube protector having an elongated tubular member designed so as to have a first end extending out of a patient's mouth when in use with the second end configured to enter the throat of the patient. The tubular member has openings at both ends and defines a substantially cylindrical cavity with an interior wall configured to hold an endotracheal tube therewithin. The tubular member has a longitudinal slit which functions to permit radial expansion of the cavity when the edges of the slit are stretched apart, for example, when a larger diameter tube is inserted into the cavity. The member is formed from a resilient material and constructed to permit stretching apart of the slit. The tubular member further includes a pair of opposed bosses formed longitudinally in the tubular member at positions adjacent to the longitudinal slit. The opposed bosses are configured to project into the cavity such that they transmit a clamping force onto the endotracheal tube when the endotracheal tube protector is contacted by the upper teeth of the patient given that the endotracheal tube protector is appropriately oriented such that the slit can face toward the roof of the patient's mouth, or rotated 90° from that position.

The endotracheal tube protector may include a third boss projecting into the cavity at a position opposing the longitudinal slit. The third boss may be formed longitudinally in the tubular member and configured to transmit clamping force when the endotracheal tube protector is in use and contacted by the lower teeth of the patient. Together, the pair of opposed bosses and the third boss are configured to provide a gap between the endotracheal tube and the interior wall of the tubular member. The gap is of sufficient size to accommodate and protect an air tube for inflation of a bulb. Protection of the air tube prevents it from being bitten or otherwise crushed in the oral cavity. The bulb is configured to protect the distal end of the endotracheal tube from blockage by bodily fluids and to prevent bodily fluids from collecting in the lungs of the patient.

The endotracheal tube protector may also include an outer flat base formed along the length of the tubular member at a position opposite the longitudinal slit. This flat base is configured to rest on a patient's tongue when the endotracheal tube protector is in use and in an orientation such that the slit is facing toward the roof of the patient's mouth. As mentioned above however, the protector according to the present invention is not limited to use in that particular orientation. Positional change is permitted by its construction or can be rotated to allow positional change not limit use and in order to additionally lessen the discomfort of the patient during intubation, the tubular member may also be tapered in a direction extending toward the back end such that the cross section of the tubular member is progressively reduced to provide less contact and pressure, when in use, against the throat and the roof of the patient's mouth. The endotracheal tube protector may also have an inwardly cut-away portion positioned and configured for allowing the endotracheal tube to bend outwardly at an angle from the endotracheal tube protector, thus providing more flexibility for the endotracheal tube to be positioned optimally in context of the endotracheal intubation system.

It is a further objective of the present invention to provide a method for positioning an endotracheal tube into the trachea of a patient. This method is performed by providing the endotracheal tube protector as described above, positioning the endotracheal tube into the cavity of the endotracheal tube protector, and inserting the back end of the endotracheal tube protector into the oral cavity of the patient such that the longitudinal slit is oriented towards the roof of the patient's oral cavity and orienting the endotracheal tube protector such that the patient's upper and lower teeth provide clamping force onto the endotracheal tube.

It is a further objective of the present invention to provide an endotracheal intubation system for providing gas delivery or a means for entrance of a probe or medical instrument into the trachea of a patient. The system includes the endotracheal tube protector as described above, an endotracheal tube, and a means for gas delivery, a probe or a medical instrument. The means for gas delivery, probe or medical instrument are configured for slidable entry into the endotracheal tube. The system may also include an air tube for inflation of a bulb which is designed to protect the distal end of the endotracheal tube from blockage by bodily fluids and to prevent bodily fluids from collecting in the lungs of the patient.

### BRIEF DESCRIPTION OF THE DRAWINGS

FIG. 1 is a cross sectional view of the endotracheal tube protector in use;

FIG. 2 is a side view of the endotracheal tube protector;

FIG. 3 is a top view of the endotracheal tube protector;

FIG. 4 is a cross sectional view of the endotracheal tube protector taken on line 3-3 of FIG. 6.

FIG. 5 is a further cross sectional view of the endotracheal tube protector taken on line 3-3 of FIG. 6, including cross sections of the air line and the endotracheal tube; and

FIG. 6 is a perspective view of the endotracheal tube protector.

FIG. 7 is a perspective view of the endotracheal tube protector.

### DETAILED DESCRIPTION OF EMBODIMENTS

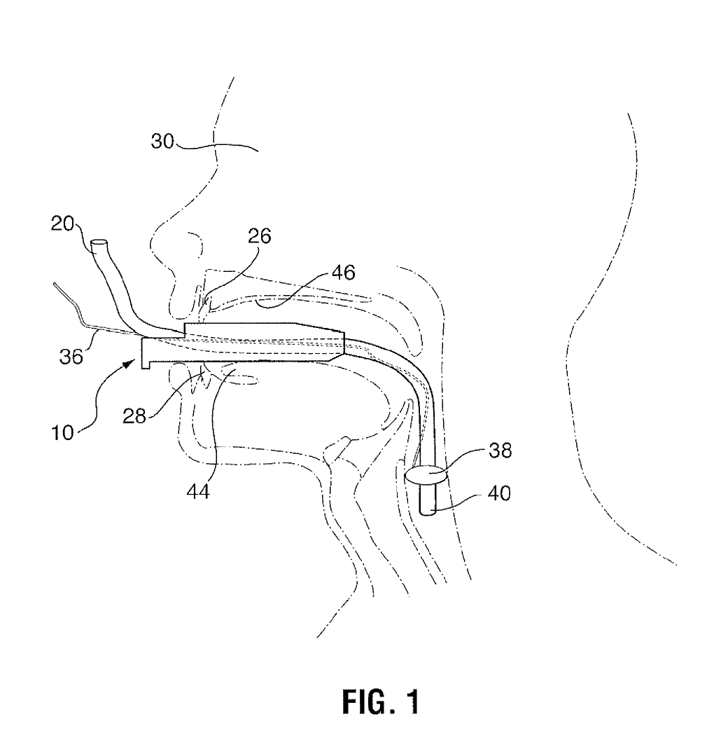

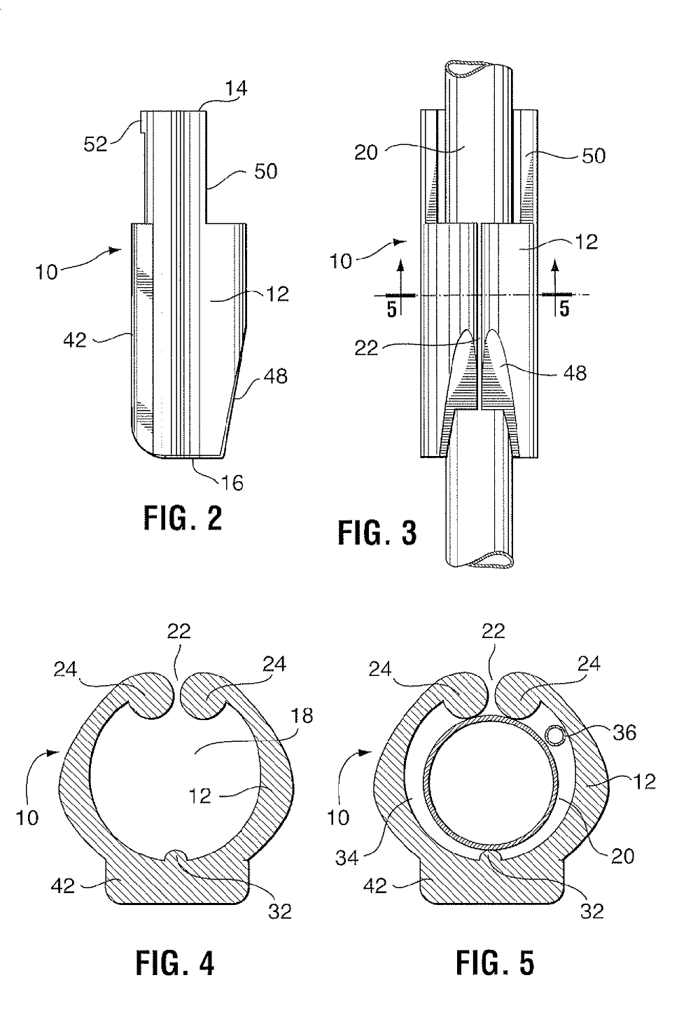

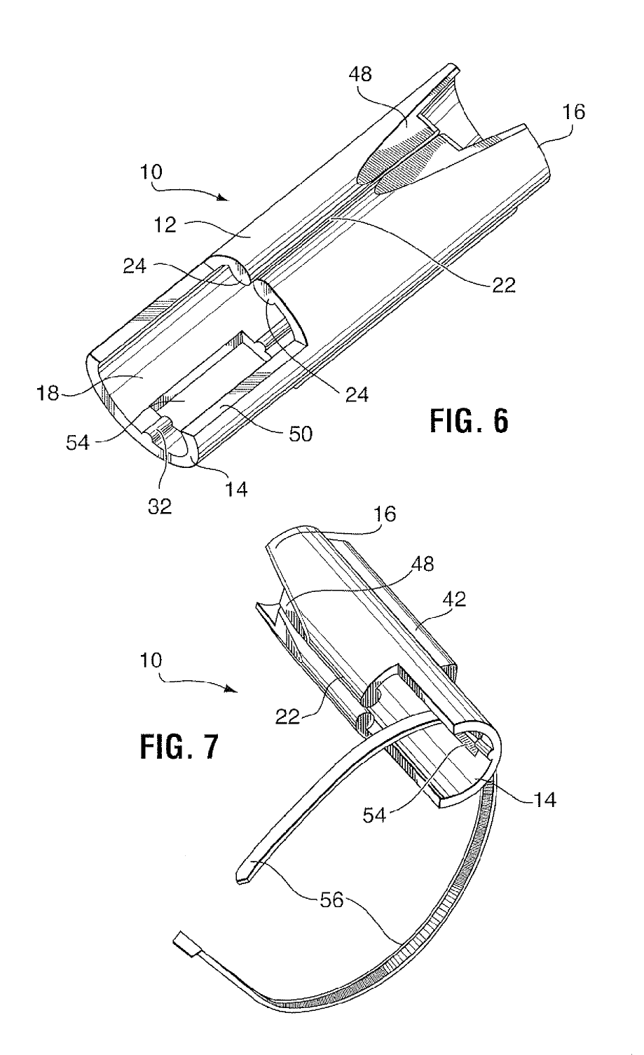

The endotracheal tube protector (10) shown in FIGS. 1 to 6, is an elongated tubular member (12) formed of resilient material. The tubular member has a first end (14) and a second end (16). The first end (14) is configured to extend from the mouth of a patient while the second end (16) is configured to enter the throat of the patient. The tubular member defines a substantially cylindrical cavity (18) with an interior wall configured to hold an endotracheal tube (20) therewithin. The tubular member (12) has a longitudinal slit (22) for inducing radial expansion of the cavity (18). The tubular member (12) further includes a pair of opposed bosses (24) formed longitudinally in the tubular member (12) at positions adjacent to the slit. The opposed bosses (24) are configured to project into the cavity (18) and configured to transmit a clamping force onto the endotracheal tube (20) when the endotracheal tube protector is contacted by the upper teeth of the patient (26) when the endotracheal tube protector is in use and oriented such that the slit faces toward the roof of the patient's mouth.

In some embodiments, the endotracheal tube protector also includes a third boss (32) projecting into the cavity (18) at a position opposing the longitudinal slit (22). This third boss (32) is formed longitudinally in the tubular member (12) and configured to transmit clamping force when the endotracheal tube protector is in use and contacted by the lower teeth (28) of the patient (30).

In some embodiments, the pair of opposed bosses (24) and the third boss (32) are configured to provide, when in use, a gap (34) between the endotracheal tube and the interior wall of the tubular member. The gap is of sufficient size to accommodate and protect against being crushed an air tube (36) for inflation of a bulb (38) which is configured to protect the distal end (40) of the endotracheal tube (20) from blockage by bodily fluids and to prevent bodily fluids from collecting in the lungs of the patient.

In some embodiments, the tubular member (12) includes an outer lower flat base (42) formed longitudinally in the tubular member (12) at a position opposite the longitudinal slit (22). The flat base (42) is configured for conforming to and resting on a patient's tongue (44).

In some embodiments, the tubular member has a taper (48) in a direction extending toward the second end such that the cross section of the tubular member (12) is progressively reduced to provide less contact and pressure, when in use, against the roof (46) of the patient's mouth and the throat of the patient, thereby improving the comfort of the patient.

In some embodiments, the first end (14) of the tubular member (12) is provided with an inwardly cut-away portion (50) extending towards the second end (16). The cut-away portion is positioned and configured to allow the endotracheal tube (20), when in position, to extend outwardly from the tubular member (12) at an angle thereto.

In some embodiments, the first end (14) of the endotracheal tube protector (10) has a slot (54) which is configured to receive a means for securing the endotracheal tube to said endotracheal tube protector. The means for securing can be a cable tie (56), elastic band, or other such device. The cable tie (56) is configured to encircle and secure the endotracheal tube (20) to the endotracheal tube protector (10) to prevent the endotracheal tube (20) from moving and dislodging from the endotracheal tube protector (10) or sliding further down the patient's trachea during use.

In some embodiments, the endotracheal tube protector (10) further includes a handle protrusion (52) extending from the first end (14) in a substantially perpendicular direction with respect to a plane defined by the length of the tubular member (12). The handle protrusion (52) is configured for grasping and facilitating removal of the endotracheal tube protector (10) from the mouth of the patient.

In some embodiments, it is advantageous to have the endotracheal tube protector (10) moulded from resilient flexible plastic material. This allows temporary spreading apart of portions of the endotracheal tube protector on either side of the slit (22) to allow insertion of endotracheal tubes of diameters ranging from about 6 millimeters to about 9.5 millimeters into the cavity (18) and return of those portions to their normal position so that the inserted endotracheal tube is held within the cavity (18). A smaller endotracheal tube protector (10) may be provided for pediatric use, for insertion of endobroncheal tubes of diameters ranging from about 4 to 6.5 millimeters. In some embodiments, the material from which the endotracheal tube protector is moulded has a Shore A durometer scale hardness from about 60 to about 90. This hardness of the material is selected to provide sufficient friction against the patient's mouth and throat that a means for securing the endotracheal tube protector from sliding down into the patient's throat is not required. It is also advantageous to select endotracheal tube protector moulding material that is sterilizable so that the endotracheal tube protector can be sterilized and re-used. In some cases, it may also be advantageous to select moulding material which is inexpensive so that the endotracheal tube protector can be designed for single use and then disposed of.

In some embodiments, the endotracheal tube protector (10) is employed in methods for intubating a patient. Such methods may be performed by positioning an endotracheal tube (20) into the cavity (18) of the endotracheal tube protector (10) and inserting the second end (16) of the endotracheal tube protector into the oral cavity of the patient such that the longitudinal slit (22) is oriented towards the roof (46) of the patient's mouth. The endotracheal tube protector is then oriented such that the patient's upper (26) and lower (28) teeth provide clamping force onto the endotracheal tube (20).

In some embodiments, the endotracheal tube protector (10) is employed in combination with other components in endotracheal intubation systems that include an endotracheal tube and a gas delivery means such as an oxygen or air delivery system, probes or medical instruments that are configured for slidable entry into the endotracheal tube. The system may also include an air tube for inflation of a bulb which is configured to protect the distal end of the endotracheal tube from blockage by bodily fluids and to prevent the bodily fluids from collecting in the lungs of the patient.

It will be seen that the present invention has been described by way of preferred embodiments of various aspects of the invention. However, it will be understood that one skilled in the art may readily depart from the embodiments described in detail herein, while still remaining within the scope of the invention as defined in this patent specification including the claims thereto.

What I claim as my invention:

1. An endotracheal tube protector for an endotracheal tube comprising:
an elongated tubular member having a first end configured to extend out of a patient's mouth when in use and a second end configured to enter the throat of said patient, said tubular member having openings in said first and second ends and defining a substantially cylindrical cavity with an interior wall configured to slidably hold an endotracheal tube therewithin, prior to application of a clamping force, said tubular member having a longitudinal slit for inducing radial expansion of said cavity, said tubular member further including a pair of opposed bosses formed longitudinally in said tubular member at positions adjacent to said slit, said opposed bosses configured to project into said cavity and configured to transmit said clamping force onto said endotracheal tube when contacted by upper teeth of said patient when said endotracheal tube protector is in use and oriented such that said slit faces toward the roof of said patient's mouth.

2. The endotracheal tube protector of claim 1 wherein said tubular member further comprises a third boss projecting into said cavity at a position opposing said longitudinal slit, said third boss formed longitudinally in said tubular member, said third boss configured to transmit clamping force when said endotracheal tube protector is in use and contacted by lower teeth of said patient.

3. The endotracheal tube protector of claim 2 wherein said pair of opposed bosses and said third boss are configured to provide, when in use, a gap between said endotracheal tube and said interior wall of said tubular member, wherein said gap is of sufficient size to accommodate and protect an air tube for inflation of a bulb, said bulb configured to protect the distal end of said endotracheal tube from blockage by bodily fluids and to prevent said bodily fluids from collecting in the lungs of said patient.

4. The endotracheal tube protector of claim 1 wherein said tubular member further comprises an outer lower flat base formed longitudinally therein at a position opposite said longitudinal slit, said lower flat base configured for conforming to and resting on said patient's tongue.

5. The endotracheal tube protector of claim 1 wherein said tubular member is tapered in a direction extending toward said second end such that the cross section of said tubular member is progressively reduced to provide less contact and pressure, when in use, against the roof of said patient's mouth and the throat of said patient, thereby improving the comfort of said patient.

6. The endotracheal tube protector of claim 1 wherein said first end of said tubular member is provided with an inwardly cut-away portion extending towards said second end, said cut-away portion positioned and configured for allowing said endotracheal tube, when in position, to extend outwardly from said tubular member at an angle to said tubular member.

7. The endotracheal tube protector of claim 6 further comprising a slot adjacent to said first end of said tubular member, said slot configured to receive therethrough a means for securing said endotracheal tube to said endotracheal tube protector.

8. The endotracheal tube protector of claim 7 wherein said means for securing is a cable tie configured to encircle and secure said endotracheal tube to said endotracheal tube protector.

9. The endotracheal tube protector of claim 1 further comprising a handle protrusion extending from said first end in a substantially perpendicular direction with respect to a plane defined by the length of said tubular member, said handle protrusion configured for grasping and facilitating removal of said endotracheal tube protector from the mouth of said patient.

10. The endotracheal tube protector of claim 1 moulded from resilient plastic material.

11. The endotracheal tube protector of claim 10 wherein said material has sufficient flexibility to allow endotracheal tubes of diameters ranging from about 6 millimeters to about 9.5 millimeters to be held in said cavity.

12. The endotracheal tube protector of claim 10 wherein said material has a Shore A durometer scale hardness from about 60 to about 90.

13. The endotracheal tube protector of claim 10 wherein said material is selected to provide sufficient friction against said patient's mouth and throat that a means for securing said tubular member from sliding down into said patient's throat is not required.

14. The endotracheal tube protector of claim 10 wherein said material is sterilizable.

15. A method for positioning of an endotracheal tube into the trachea of a patient comprising:
providing a endotracheal tube protector comprising an elongated tubular member having an first end configured to extend out of said patient's mouth when in use and a second end configured to enter the throat of said patient, said tubular member having openings in said first and second ends and defining a substantially cylindrical central cavity with an interior wall configured to hold an endotracheal tube therewithin, said tubular member having a longitudinal slit and a pair of opposed bosses formed longitudinally in said tubular member at positions adjacent to said slit, said opposed bosses configured to project into said cavity and configured to transmit clamping force onto said endotracheal tube when in use and when contacted by upper teeth of said patient when said endotracheal tube protector is in use and oriented such that said slit faces toward the roof of said patient's mouth;
positioning said endotracheal tube into said cavity of said endotracheal tube protector; and
inserting said second end of said endotracheal tube protector into the oral cavity of said patient such that said longitudinal slit is oriented towards the roof of said patient's oral cavity and said endotracheal tube protector is oriented such that said patient's upper and lower teeth provide said clamping force onto said endotracheal tube.

16. An endotracheal intubation system for providing gas delivery or a means for entrance of a probe or medical instrument into the trachea of a patient comprising:
an endotracheal tube;
a endotracheal tube protector comprising an elongated tubular member having a first end configured to extend out of said patient's mouth when in use and a second end configured to enter the throat of said patient, said tubular member having openings in said first and second ends and defining a substantially cylindrical central cavity with an interior wall configured to hold said endotracheal tube therewithin, said tubular member having a longitudinal slit and a pair of opposed bosses formed longitudinally in said tubular member at positions adjacent to said slit, said opposed bosses configured to project into said cavity and configured to transmit a clamping force onto said endotracheal tube when in use and when contacted by upper teeth of said patient when said endotracheal tube protector is in use and oriented such that said slit faces toward the roof of said patient's mouth; and
a gas delivery means, probe or medical instrument configured for slidable entry into said endotracheal tube.

17. The endotracheal intubation system of claim 16 further comprising an air tube for inflation of a bulb, said bulb configured to protect the distal end of said endotracheal tube from blockage by bodily fluids and to prevent said bodily fluids from collecting in the lungs of said patient.
